(19) United States
(12) Patent Application Publication
(10) Pub. No.: US 2011/0034941 A1
(43) Pub. Date: Feb. 10, 2011

(54) Surgical Instrument for Hernia Repair and Method

(76) Inventors: Joseph Iraci, New Rochelle, NY (US); Mark P. Zoland, Scarsdale, NY (US)

Correspondence Address: GORDON & JACOBSON, P.C., 60 LONG RIDGE ROAD, SUITE 407, STAMFORD, CT, 06902, US

(21) Appl. No.: 12/538,264

(22) Filed: Aug. 10, 2009

(51) Int. Cl. A61B 17/03

(52) U.S. Cl. 606/151

(57) ABSTRACT

A surgical instrument for storing, deploying, manipulating, and securing a surgical mesh at a surgical site is provided. The surgical instrument includes an elongate member, a drive member extending through an interior channel defined by the elongate member, and proximal and distal fixation members for securing the surgical mesh at a surgical site. The surgical instrument in initially provided in a loaded configuration in which the mesh and the proximal and distal fixation members are disposed inside a distal interior chamber at the distal end of the elongate member and detachably coupled to the drive member. The surgical instrument is configurable to multiple deployment configurations to deploy the surgical mesh at the surgical site and to attach the proximal and distal fixation members to the mesh and to tissue at the surgical site for securing the mesh at the surgical site.

### BACKGROUND OF THE INVENTION

1. Field of the Invention

This invention relates broadly to surgical instruments. More particularly, this invention relates to surgical instruments for deploying surgical meshes for hernia repair at a surgical site.

2. State of the Art

Hernias are caused by abnormal defects, tears, or natural openings in membranes, layers of muscle, and/or bone in the body. Such defects may weaken the structural integrity of the defect area and can permit migration of adjacent body structures and/or surrounding tissue (e.g., through an opening), which can result in serious and quite painful symptoms. Hernias are generally classified as direct inguinal hernias, indirect inguinal hernias, or femoral hernias. In direct and indirect inguinal hernias, a portion of the intestine often protrudes through a defect in the supporting abdominal wall. In a femoral hernia, a portion of the intestine is often forced through the femoral ring into the femoral canal.

Historically hernias have been treated by providing an incision through the abdominal wall and retracting layers of healthy tissue to expose the defect. The defect was often repaired by sewing strong surrounding muscle over the defect. Alternatively, the defect was often repaired by covering the defect with a mesh (or other implant). Patients undergoing such procedures typically experienced at least a week of painful recovery time. More recently, laparoscopic and endoscopic methods have been utilized in which a scope is inserted through a cannula positioned within the abdominal wall to provide an intra-tissue view adjacent the hernia. Additional tools are then inserted through additional cannulae extending within the abdominal wall for introducing, grasping, and setting a surgical mesh or other implantable insert at the surgical site of the hernia. This process generally requires viewing the surgical site with the scope through a first port, introducing the mesh with a deployment apparatus through a second port, and then utilizing additional instruments, including a grasper, via a third port to manipulate the inserted mesh or other implantable insert over the hernia area and to optionally secure it thereto (e.g., with tacks or sutures).

### SUMMARY OF THE INVENTION

The invention provides a surgical instrument for storing, deploying, manipulating, and securing a surgical mesh to tissue adjacent a hernia defect (referred to herein as a “surgical site”). The surgical instrument includes an elongate member which defines an interior channel extending therethrough to an interior distal chamber. A drive member extends through the channel of the elongate member. A surgical mesh together with proximal and distal fixation members are loaded into the distal chamber with the proximal and distal fixation members detachably coupled to the distal end of the drive member. An opening at the distal end of the elongate member provides a passageway for deployment of the surgical mesh and fixation members loaded in the distal chamber at the surgical site and for driving the fixation members into tissue at the surgical site for securing the surgical mesh at the surgical site.

The surgical mesh and proximal and distal fixation members may be pre-loaded in the distal chamber by the manufacturer, distributor or other non-user, or alternatively may be loaded therein by a surgeon or other user. In the preferred embodiment, the mesh and fixation members are loaded into the distal chamber by advancing the drive member distally relative to the elongate member in order to expose a distal portion of the drive member. The surgical mesh is helically coiled around the exposed distal portion of the drive member. The proximal and distal fixation members are detachably coupled to each other and to the distal end of the drive member in an end-to-end configuration. The distal fixation member is attached to a section of the surgical mesh. After coupling the mesh and fixation members to the drive member, the drive member is retracted relative to the elongate member such that the mesh and the fixation members are housed inside the distal chamber. In the preferred embodiment, when loaded inside the distal chamber, the fixation members are positioned end-to-end within interior cylindrical space defined by the helically-coiled mesh and aligned to the longitudinal axis of the drive member.

With the surgical mesh and fixation members loaded inside the distal chamber, the distal end of the instrument is positioned adjacent the surgical site to deploy and attach the surgical mesh to the surgical site. Initially, a force is applied to the drive member to advance the drive member distally relative to the elongate member such that at least a portion of the distal fixation member and possibly the section of mesh attached thereto pass through the opening leading from the distal chamber to a position outside of the elongate member, referred to as the first deployment configuration. In the first deployment configuration, the drive member is manipulated by the surgeon to drive the distal fixation member into first tissue at the surgical site to thereby secure the section of surgical mesh attached thereto to the first tissue at a position dictated by the surgeon. Because the distal and proximal fixation members are loaded in an end-to-end arrangement, the driving action of the drive member is transmitted through the proximal fixation member when driving the distal fixation member. After the distal fixation member is secured to the surgical site, the distal fixation member is decoupled from the instrument (e.g., detached from the proximal fixation member).

The elongate member is then moved relative to the drive member (by advancing the drive member distally relative to the elongate member, or by retracting the elongate member proximally relative to the drive member or any combination thereof) such that the entire surgical mesh passes through the opening leading from the distal chamber to a deployed position outside of the elongate member, referred to herein as a second deployment configuration. In the second deployment configuration, the drive member is preferably in a fully extended position relative to the elongate member and the proximal fixation member is detachably coupled to the distal end of the drive member. In addition, in the second deployment configuration, the drive member is utilized to secure the proximal fixation member to the fully deployed surgical mesh at second tissue at the surgical site, preferably at a location offset from the first tissue. In the preferred embodiment, a finger grip and a palm grip disposed on respective outer surfaces of the elongate member and the drive member function as a stop to prevent the drive member from being distally advanced beyond its position relative to the elongate member in the second deployment configuration.

In the preferred embodiment, the surgeon deploys the surgical mesh from the distal chamber by manipulating the drive member to unfurl the helically coiled mesh in a controlled manner with the mesh section secured to the first tissue. Such controlled unfurling allows the surgeon to place the surgical mesh into a desired position adjacent the surgical site to cover the hernia defect. In the second deployment configuration, with the surgical mesh positioned adjacent the surgical site and covering the hernia defect, the drive member is manipulated by the surgeon to drive the proximal fixation member through a section of the surgical mesh overlying the second tissue at the surgical site offset from the distal fixation member (preferably on the other side of the defect) and into such second tissue to thereby secure the surgical mesh to the second tissue. In this manner, the surgical mesh is secured at the surgical site by distal and proximal fixation members that anchor spaced apart sections of the surgical mesh to first and second tissues at the surgical site.

In the preferred embodiment, the elongate member is a tube having a diameter which preferably does not exceed 5 mm. The drive member is also preferably a mandrel which is longitudinally translatable and rotatable relative to the elongate member.

According to one aspect of the invention, the drive member is rotatably coupled to the elongate member such that rotation of the drive member relative to the elongate member causes longitudinal translation of the drive member relative to the elongate member.

According to yet another aspect of the invention, the proximal and distal fixation members are both longitudinally aligned about a longitudinal axis of the drive member and connected in an end to end configuration in the loaded and first deployment configurations such that a longitudinal force applied to the proximal end of the drive member is transmitted through the proximal fixation member to the distal fixation member.

Additional objects and advantages of the invention will become apparent to those skilled in the art upon reference to the detailed description taken in conjunction with the provided figures.

### BRIEF DESCRIPTION OF THE DRAWINGS

FIG. 1A is a broken front view of the elongate member and drive member of the invention in a loaded configuration.

FIG. 1B is a broken cutaway view of the distal end of the elongate member of the invention in the loaded configuration.

FIG. 1C is a broken cutaway view of a proximal portion of the elongate member of the invention in the loaded configuration.

FIG. 2A is a broken front view of the elongate member and drive member of the invention in a first deployment configuration.

FIG. 2B is a broken cutaway view of the distal end of the elongate member of the invention in the first deployment configuration.

FIG. 3A is a broken front view of the elongate member and drive member of the invention between the first deployment configuration and a second deployment configuration.

FIG. 3B is a broken cutaway view of the distal end of the elongate member of the invention between the first and second deployment configurations.

FIG. 4A is a broken front view of the elongate member and drive member of the invention in the second deployment configuration.

FIG. 4B is a broken cutaway view of the distal end of the elongate member of the invention in the second deployment configuration.

### DETAILED DESCRIPTION OF THE PREFERRED EMBODIMENTS

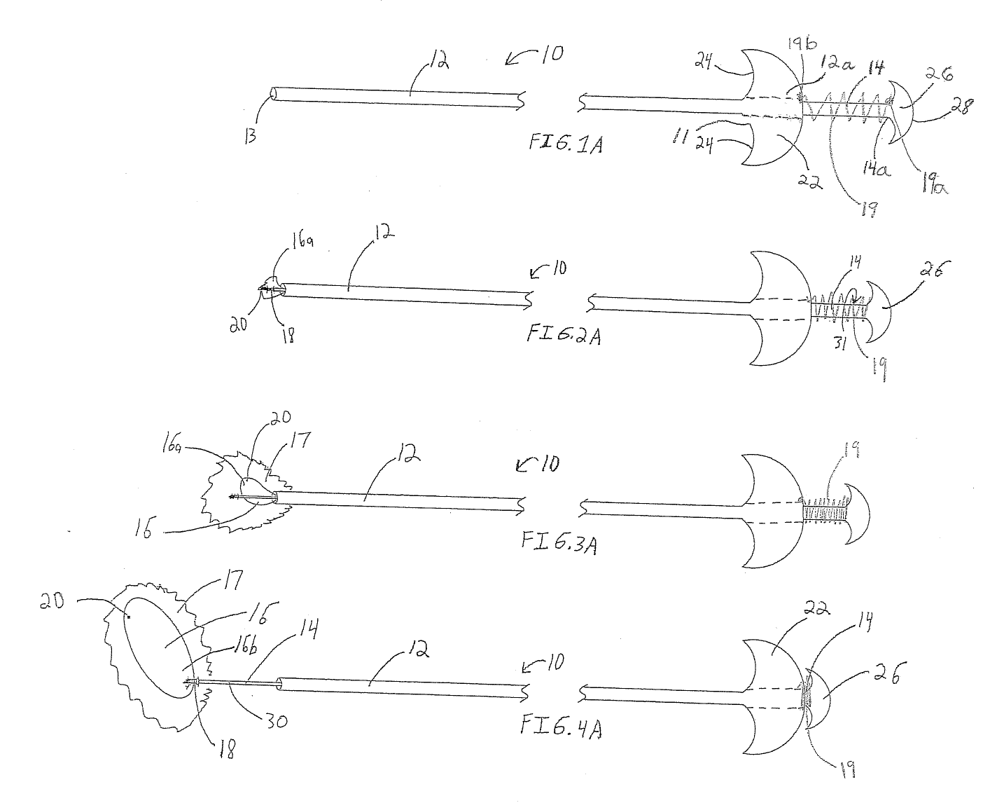

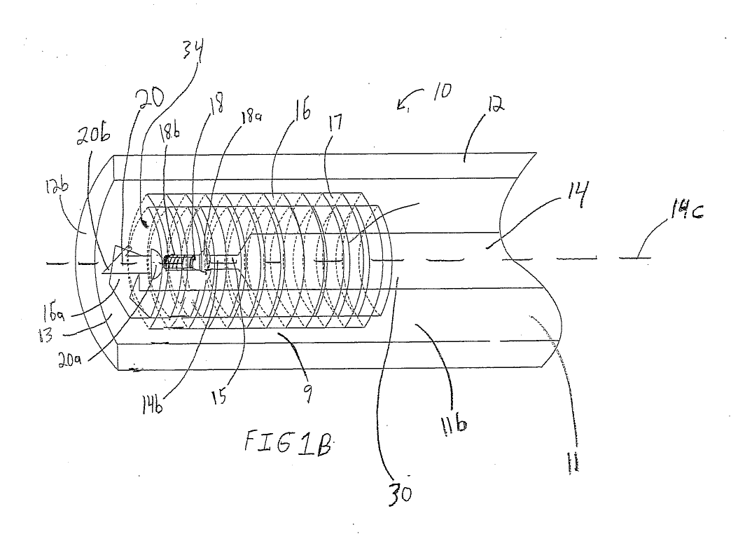

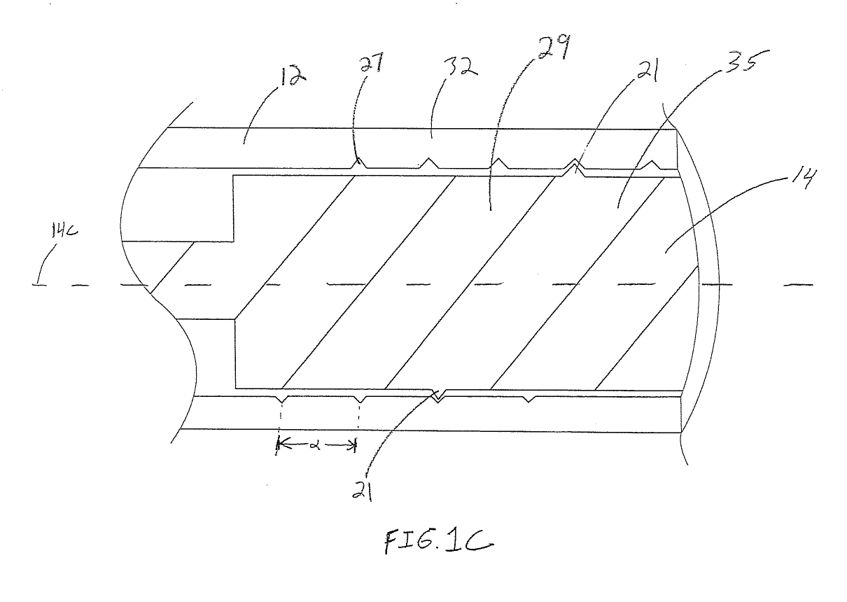

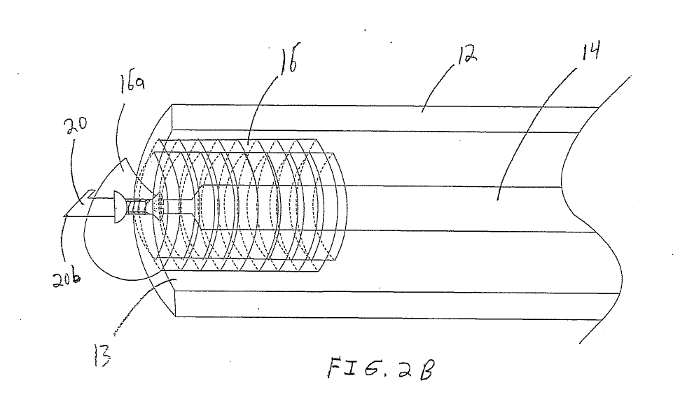

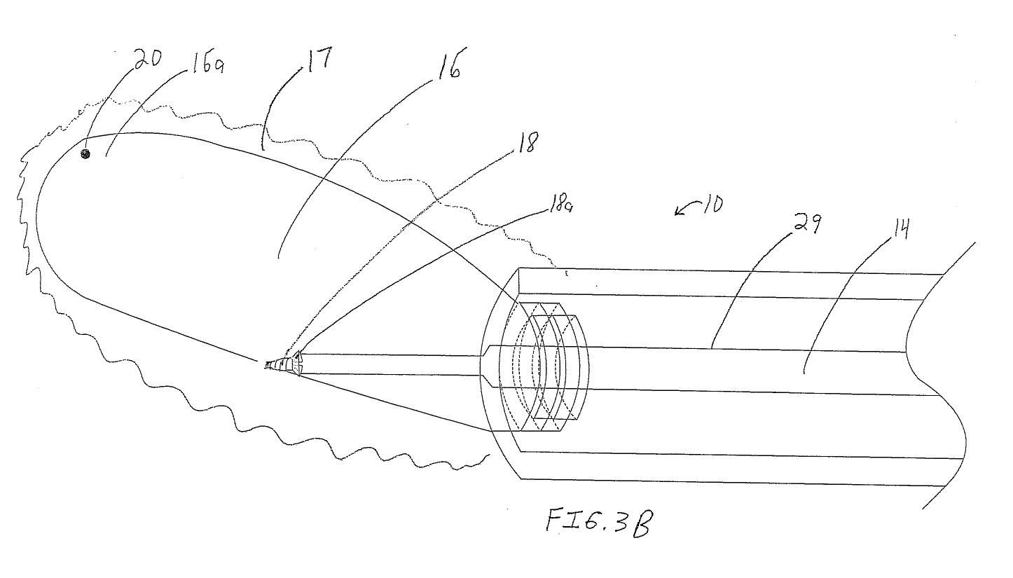

Turning to FIGS. 1A and 1B, a surgical instrument 10 for storing, deploying, manipulating, and securing a surgical mesh 16 at a surgical site 17 is shown. The surgical instrument 10 includes an elongate member 12 having proximal and distal ends 12a, 12b. The elongate member 12 defines an interior channel 11 (FIG. 1B) which extends therethrough to an interior distal chamber 9 adjacent the distal end 12b of the elongate member 12. A drive member 14 extends through the channel 11 of the elongate member 12 and includes a proximal end 14a (FIG. 1A) and a distal end 14b (FIG. 1B). A surgical mesh 16, together with proximal and distal fixation members 18, 20, are provided in the distal chamber 9 for deployment at the surgical site 17 (FIG. 3A) as further discussed below. The proximal and distal fixation members 18, 20 are detachably coupled to the distal end 14b of the drive member 14 and to each other, preferably in an end to end arrangement and in alignment with a longitudinal axis 14c of the drive member 14. The loading, deployment, and attachment of the mesh 16 and fixation members 18, 20 at the surgical site is further discussed below with respect to FIGS. 2A-4B following a description of each of the components of the instrument 10.

Still referring to FIGS. 1A and 1B, the elongate member 12 is preferably a tube with an outer diameter preferably not exceeding 5 mm. The proximal end 12a of the elongate member 12 preferably includes a finger grip 22 defining finger holds 24 positioned to allow a surgeon to grasp the finger grip 22 with at least one finger. The finger grip 22 may be constructed in other configurations which facilitate manipulation of the drive member 14 relative to the elongate member 12 as further discussed below.

The drive member 14 is preferably realized by a mandrel 29 (FIG. 1C). A portion of the mandrel 29 includes guides 21 that protrude from the outer surface of the drive member 14 and mate with a helical groove 27 defined by a preferably proximal portion 32 of the elongate member 12 as shown in FIG. 1C. The interface between the guides 21 and the helical groove 27 causes the drive member 14 to rotate relative to the elongate member 12 when a user provides an axial force on the drive member 14. It will be appreciated by those skilled in the art that the pitch a of the helical groove 27 and the particular structure of the guide-groove interface govern the amount of axial force and rotational force translated to the drive member 14 in response to an axial force applied to the palm grip 26 (further discussed below) at the proximal end 14a of the drive member 14. The guides 21 and the helical groove 27 are preferably disposed along the proximal portion 32 of the elongate member 12 and the proximal portion 35 of the drive member 14, and may even be disposed adjacent the proximal end 12a of the elongate member 12. It is also contemplated that the groove 27 and/or guides 21 can be defined by sleeve inserts that are secured to the respective parts.

The elongate member 12 is also preferably coupled to the drive member 14 via a spring 19 which attaches to the drive member 14 at a proximal end 19a, and to the elongate member 12 at a distal end 19b. The spring 19 functions to bias the drive member 14 in the retracted position of FIG. 1A (with the distal fixation member 20 inside of the distal chamber 9) and prevents inadvertent movement of the elongate member 12 and drive member 14 relative to each other when the instrument 10 is advanced through a port or cannula to a surgical site. The spring 19 is also coupled between the elongate member 12 and drive member 14 to prevent complete separation of the elongate and drive members 12, 14 from each other. In addition or alternatively, other structure may be employed for this purpose, such as, for example, one or more interfering collars, flanges, or bushings attached to the drive member 14 and/or elongate member 12

As described above, the drive member 14 can be rotated and translated relative to the elongate member 12. The proximal end 14a of the drive member 14 preferably includes a palm grip 26 defining a palm seat 28 shaped and positioned to allow a surgeon to grasp the palm grip 26 with a palm of a hand while simultaneously grasping the finger grip 22 with at least one finger. The palm grip 26 is thus preferably offset from the finger grip 22 when the drive member 14 is fully retracted relative to the elongate member 12 to allow a surgeon to properly grasp the palm grip 26 simultaneously with the finger grip 22 for operation thereof while providing enough stroke length to the drive member 14 relative to the elongate member 12 to deploy the surgical mesh 16 as further discussed below. The palm grip 26 may alternatively be constructed in other shapes and sizes which facilitate the application of a longitudinal or rotational force thereto to cause translation and/or rotation) of the drive member 14 relative to the elongate member 12. The finger grip 22 and the palm grip 26 thus together function as a handle for grasping and orienting the instrument 10, and for moving the drive member 14 and the elongate member 12 relative to each other. As shown in FIGS. 1A and 1B, when the drive member 14 is fully retracted relative to the elongate member 12, the palm grip 26 is disposed furthest from the finger grip 22 and the distal portion 30 (FIG. 1B) of the drive member 14 is disposed inside the distal portion 11b of the channel 11.

The distal end 14b (FIG. 1B) of the drive member 14 includes a drive tip 15 which has a relatively smaller diameter than the distal portion 30 of the drive member 14. The drive tip 15 is detachably coupled to the proximal fixation member 18 as further discussed below in order to transmit forces from the drive member 14 to the proximal and distal fixation members 18, 20 to facilitate manipulation thereof.

The surgical mesh 16 is preferably provided in a helically coiled configuration around the drive member 14 inside the interior chamber 9. In this configuration, as best shown in FIG. 1B, the coiled surgical mesh 16 is also coiled around the proximal and distal fixation members 18, 20 and defines a cylindrical space 34 within the chamber 9 distal of the distal end 14b of the drive member 14.

The mesh 16 also preferably includes a plurality of openings 17 which allow for tissue ingrowth through the mesh 16 once the mesh 16 is deployed at the surgical site 17. The surgical mesh 16 is preferably made from a pliable tissue fabric which is biased toward a flat configuration (e.g., the mesh 16 is sufficiently pliable to allow it to be rolled around the drive member 14 into the shape of a cylinder or helical coil suitable for entry into an opening 13 (also referred to as a passageway herein) to the chamber 9 as shown in FIG. 1B), but also sufficiently elastic to automatically return to a flat configuration with sufficient area to extend across a defect area once deployed at the surgical site 17.

The surgical mesh 16 may be formed from a sheet of knitted polypropylene monofilament mesh fabric such as MARLEX mesh available from C.R. Bard, Inc. The mesh 16 may be made from other materials which are suitable for tissue reinforcement and/or closure of a defect area, including PROLENE, MERSELENE, DACRON, TEFLON textile based meshes, microporous polypropylene sheeting CELGARD, and expanded PTFE (GORETEX) as discussed in U.S. Pat. No. 6,267,772 to Mulhauser et al., which is herein incorporated by reference in its entirety. When the surgical mesh 16 is implanted at the surgical site 17, it may stimulate an inflammatory reaction which promotes rapid tissue growth into and around the mesh structure.

The proximal fixation member 18 is preferably a screw which includes a proximal head 18a and distal threads 18b. The proximal head 18a of the screw 18 is detachably coupled to the drive tip 15 of the drive member 14, preferably by a hex driver and hex slot interface supplemented with an adhesive (e.g., a medical-grade adhesive such as a silicone, alpha-cyanoacrylates, etc.) which is solvent-free and nontoxic once it is cured, and which has been tested for proper biocompatibility (e.g., USP or Class VI standard to ISO-10993.). The hex slot (not shown) is defined within the proximal head 18a of the screw 18, and the hex driver (not shown) is defined at the distal end 14b of the drive member 14. The detachable coupling of the proximal head 18a of the proximal fixation member 18 to the distal end 14b of the drive member 14 allows for proximal and distal movement of the screw 18 and rotation of the screw 18 by the drive member 14.

The distal fixation member 20 is preferably a tack which includes a proximal head 20a and a distal barb 20b. The distal barb 20b is pointed and pierced through a section 16a of the surgical mesh 16. The proximal head 20a of the distal fixation member 20 is detachably coupled to the distal end 18b of the proximal fixation member 18, preferably also by an adhesive, a releasable bond, or a frangible link.

Regarding assembly, the surgical instrument 10 is preferably provided with the drive member 14 pre-assembled inside the elongate member 12 and extending through the channel 11, and with the finger grip 22 and the palm grip 26 disposed outside of and proximal to the elongate member 12 as shown in FIG. 1A. The surgical instrument 10 is preferably pre-loaded with the fixation members 18, 20 and surgical mesh 16 detachably coupled to the distal end 14b of the drive member 14 as shown in FIG. 1B).

In yet another alternative, the surgical instrument 10 can be initially provided with the fixation members 18, 20 and surgical mesh 16 detached from the elongate member 12, and these components may be attached to the instrument 10 and loaded into the chamber 9 as follows. The drive member 14 is advanced distally relative to the elongate member 12 by applying an axial force to the palm grip 26 to fully expose the distal portion 30 of the drive member 14. The proximal fixation member 18 is then detachably coupled to the distal end 14b of the drive member 14 in the manner discussed above (e.g., hex driver/hex slot interface plus an adhesive, a bond, or a frangible link). The surgical mesh 16 is helically coiled around the distal portion 30 of the drive member 14 and the distal fixation member 20 is attached to the section 16a of mesh 16 via the barb 20b. The distal fixation member 20 is detachably coupled to the proximal fixation member 18 as discussed above. After coupling the mesh 16 and fixation members 18, 20 to the distal portion 30 of the drive member 14, the drive member 14 is retracted proximally relative to the elongate member 12 to fully load the mesh 16 and fixation members 18, 20 (including the distal barb 20b) through the opening 13 and into the chamber 9 at the distal end 12b of the elongate member 12 to achieve the loaded configuration of FIGS. 1A, 1B, and 1C.

As shown in FIG. 1B, in the loaded configuration of the instrument 10, the proximal and distal fixation members 18, 20 are fully disposed inside the distal chamber 9 in an end-to-end configuration, and are preferably in alignment with a longitudinal axis 14c of the drive member 14. In addition, it is noted that in the loaded configuration, the proximal and distal fixation members 18, 20 are preferably disposed in the cylindrical space 34 defined by the helically coiled mesh 16 and positioned adjacent the distal end 14b of the elongate member 14.

With the surgical instrument 10 in the loaded configuration of FIGS. 1A, 1B, and 1C, the instrument 10 is distally advanced through a cannula and/or port in the body to a position adjacent the surgical site 17. Once the instrument 10 is positioned adjacent the surgical site 17, the instrument 10 is manipulated to a first deployment configuration as follows. The finger grip 22 and palm grip 26 are grasped by the surgeon's hand and manipulated to apply an axial force which causes the drive member 14 to advance distally relative to the elongate member 12 and to rotate in the direction of the arrow 31 of FIG. 2A. The rotation and translation of the drive member 14 is controlled until the distal fixation member 20 is at least partially exposed outside of the elongate member 12.

Turning now to FIGS. 2A and 2B, the instrument 10 has been manipulated to the first deployment configuration by distally advancing the drive member 14 relative to the elongate member 12 (FIG. 2A). As shown in FIG. 2B, in the first deployment configuration, the distal fixation member 20 and the distal section 16a of mesh 16 are both preferably at least partially exposed, having passed through the opening 13 at the distal end 12b of the elongate member 12.

It will be appreciated that, with the distal end 12b of the elongate member 12 positioned adjacent the tissue at the surgical site 17 in the loaded configuration, the drive member 14 may be manipulated to drive the barb 20b of the distal fixation member 20 and the section 16a of surgical mesh 16 into tissue at the surgical site 17. The drive member 14 may also be pushed in the distal direction to further advance the barb 20b into tissue if necessary. It will be appreciated that the respective alignment of the proximal and distal fixation members 18, 20 about the longitudinal axis 14c allows longitudinal drive forces supplied to the drive member 14 to be transmitted through the proximal fixation member 18 to the distal fixation member 20 for distal advancement thereof into the tissue.

Once the distal fixation member 20 and section 16a of surgical mesh 16 is attached at the surgical site, the elongate member 12 is retracted proximally relative to the drive member 14 by pulling the finger grip 22 proximally and pushing on the palm grip 26. Such reversed operations will cause the drive member 14 to rotate in the opposite direction and move proximally relative to the elongate member 12. The barb 20b, now stuck in tissue, will resist the proximal and rotational movement of the elongate member 14 (to which it is detachably coupled via the proximal fixation member 18). Separation of the distal fixation member 20 from the proximal fixation member 18 will thus occur when the force between them is sufficient to overcome the adhesive bond between them. It is noted that at this point, the proximal fixation member 18 is preferably not detached from the distal end 14b of the elongate member 14. Thus, it will be appreciated that the detachable coupling between the proximal fixation member 18 and the distal end 14b of the drive member 14 should require higher levels of tension and torsion to cause detachment than the levels required to cause detachment of the distal fixation member 20 (e.g., so that the distal fixation member 20 can be detached without separating the proximal fixation member 18 from the drive member 14). As discussed above, this may be accomplished by using a hex driver and hex slot coupling or other similar coupling as well as an adhesive bond between the proximal fixation member 18 and the distal end 14b of the elongate member 14, and a less resilient adhesive bond between the distal fixation member 20 and the proximal fixation member 18.

With the distal fixation member 20 separated from the proximal fixation member 18, the drive member 14 is advanced distally to configure the instrument 10 in a second deployment configuration (e.g., by squeezing the finger grip 22 and palm grip 26). As the drive member 14 is advanced distally, the drive member 14 rotates and the remainder of the surgical mesh unfurls in a controlled manner and deploys through the chamber 9 and out the opening 13 with the mesh section 16a secured to first tissue as shown in FIGS. 3A and 3B. Such controlled unfurling allows the surgeon to place the surgical mesh 16 into a desired position adjacent the surgical site 17 to cover the hernia defect. It will be appreciated that the surgical mesh 16 may thus be deployed in a controlled manner based upon the degree and force or speed with which the drive member 14 is advanced relative to the elongate member 12.

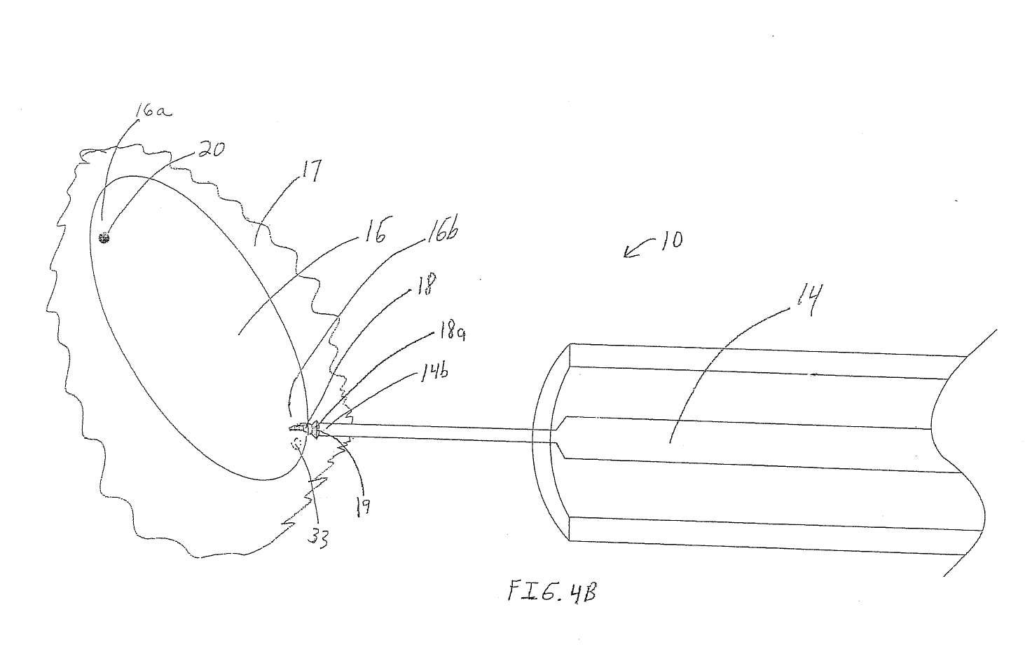

Turning to FIGS. 4A and 4B, the surgical instrument 10 is shown in a second deployment configuration in which the proximal fixation member 18 is detachably coupled to the distal end 14b of the drive member 14 and positioned distally relative to the distal end of the elongate member 12. The surgical mesh 16 is fully deployed from the instrument 10 at the surgical site 17. In this second deployment configuration, the palm grip 26 and finger grip 22 preferably function as a stop to prevent the drive member 14 from being further distally advanced relative to the elongate member 12, which is intended to prevent injury or trauma to the patient.

In the second deployment configuration, the drive member 14 is used to attach the proximal fixation member 18 to a section 16b of the surgical mesh 16, preferably at a location offset from the distal fixation member 20 (e.g., at location 33 as depicted in FIG. 4B), and secure both the proximal fixation member 18 and the surgical mesh section 16b attached thereto to tissue at the surgical site 17. As the proximal fixation member 18 is preferably a screw as discussed above, it may be screwed into adjacent tissue, bone, or ligaments as needed. It will be appreciated that the detachable coupling of the proximal fixation member 18 to the distal end 14b of the drive member 14 (e.g., via a hex driver and slot as discussed above) will facilitate the transmission of rotational and axial forces from the drive member 14 to the proximal fixation member 18.

Once the proximal fixation member 18 is fully inserted at the surgical site, the drive member 14 may be proximally retracted relative to the elongate member 12 by pulling on the palm grip 26 and pushing the finger grip 22. Proximal translation of the drive member 14 relative to the elongate member 12 will disconnect the tip 15 of the drive member 14 from the proximal fixation member 18 and break any adhesive or mechanical bond therebetween.

With the surgical mesh 16 fully deployed and the proximal and distal fixation members 18, 20 securing the mesh 16 to the surgical site 17, the surgical instrument 10 is then removed from the surgical site 17 and additional instrumentation may be used to stitch retracted tissue over the mesh 16 and surgical site 17. It will be appreciated that the surgical instrument 10 allows for the application, manipulation, and securing of a surgical mesh 16 with multiple fixation members using a single instrument in a single port or cannula.

It will be appreciated that while the distal fixation member 20 is preferably a tack which is easily inserted into soft tissue (e.g., muscle which supports and moves bones, tendons which connect muscles to bones, ligaments which connect bones to bones, synovial tissue, fascia, or other structures such as nerves, blood vessels, and fat), the proximal fixation member 18 may be, as discussed above, a screw which can be driven by the drive member 14 into hard tissue (e.g., cartilage and bone).

It will be appreciated that various deployment mechanisms can be used to deploy the surgical mesh 16 from the chamber 9 of the elongate member 12. For example, the material of the surgical mesh 16 may have shape memory with an inherent bias that aids in self-deployment of the surgical mesh from the elongate member 12. The fully-deployed configuration of the shape-memory mesh can be substantially flat to aid in covering the hernia defect at the surgical site 17.

The instrument 10 is preferably used in conjunction with an optical scope to help facilitate deployment, placement and fixation of the surgical mesh 16 at the surgical site 17. While other methodologies known in the art generally utilize multiples tools to locate, deploy and fix a surgical mesh at a surgical site (e.g., a first device which introduces the mesh, a second device which grasps the mesh and unfolds it and/or spreads it out over the defect area, and a third device which secures the mesh at the surgical site), it will be appreciated that the instrument 10 of the invention functions as the placement, grasper, and fixation tool at the surgical site 17, and thus improves efficiency and only requires the use of one or two ports in the patient. It is noted that other instruments such as laparoscopic graspers and the like can also be used in conjunction with the instrument 10 to aid in positioning the surgical mesh at the surgical site if necessary.

In an alternate embodiment, the fixation members 18, 20 and drive member 14 may be provided as a single piece of formed material with frangible sections separating each component. Each frangible section can support a different tensile and/or torsional forces as required for the driving forces that are needed to secure the distal and proximal fixation members to tissue at the surgical site. In this manner, the distal fixation member 20 may be frangibly coupled to the proximal fixation member 18 and designed to separate at a given force, and the proximal fixation member 18 may be frangibly coupled to the distal end 14b of the drive member 14 and designed to separate therefrom at a significantly higher force).

In yet another embodiment, the fixation members and drive members may be coupled with dissolvable bonds. A first dissolvable bond may be used between the driver member and first fixation member, and a second dissolvable bond may be used between the first and second fixation member. To release the first dissolvable bond, a first agent is irrigated through the elongate member or via a secondary conduit to the interface of the first and second fixation members. The first agent does not affect the second dissolvable bond. To release the second dissolvable bond, a second agent is irrigated to the interface of the proximal fixation member and the driver member.

In alternative embodiments, the drive member 14 and elongate member 12 may be provided with male and female threads coupled with an appropriate pitch for partially converting torque and rotation into longitudinal force and translation (e.g., such that torque applied to the drive member causes longitudinal translation and rotation of the drive member relative to the elongate member). In such embodiments, the drive member 14 may be rotated and distally translated relative to the elongate member 12 by simply applying torque to the palm grip 26.

There have been described and illustrated herein several embodiments of a surgical instrument for deploying a surgical mesh. While particular embodiments of the invention have been described, it is not intended that the invention be limited thereto, as it is intended that the invention be as broad in scope as the art will allow and that the specification be read likewise. Thus, while particular surgical meshes have been disclosed, it will be appreciated that other types of surgical meshes and other pliable surgical inserts may be used as well. In addition, while particular shapes of surgical meshes have been disclosed, it will be understood that various other shapes, including, elliptical, square, and rectangular shapes, can be used. Also, while an elongate member and a drive member are preferably mandrel shaped, it will be recognized that other shapes may be utilized. Furthermore, while a finger grip and palm grip have been disclosed, it will be understood that other types of hand grips may similarly be used. Moreover, while particular loading and deployment configurations have been disclosed, it will be appreciated that other configurations could be used as well. While particular types of fixation members, adhesive bonds, and detachable coupling structures have been disclosed, it will be appreciated that other types of fixation members, adhesive bonds, and detachable coupling structures may be utilized. While deployment of a surgical mesh has been disclosed using a drive member with a particular structure, it will be appreciated that other structures of drive members could be used such as a flange at the distal end of the drive member to facilitate removal of the surgical mesh through retraction of the elongate member relative to the drive member. Moreover, while particular drive mechanisms have been disclosed for effectuating desired movement of the drive member (e.g., translation and rotation) in accordance with user input for deployment and fixation of the surgical mesh, it will be appreciated that other suitable drive mechanisms can be used as well for this purpose. It will therefore be appreciated by those skilled in the art that yet other modifications could be made to the provided invention without deviating from its spirit and scope as claimed.

What is claimed is:

1. A surgical instrument for deploying and securing a surgical mesh at a surgical site, the surgical instrument comprising:
an elongate member defining an interior channel leading to a distal chamber;
a drive member extending through said channel of said elongate member, said drive member having a proximal end and a distal end;
a proximal fixation member for securing said surgical mesh at the surgical site; and
a distal fixation member for securing said surgical mesh at the surgical site,
wherein, in a loaded configuration, said mesh is disposed in a helically coiled configuration in said distal chamber and said distal and proximal fixation members are disposed in an end-to-end arrangement, and
wherein, said proximal fixation member is detachably coupled to said distal end of said drive member and said distal fixation member is detachably coupled to said proximal fixation member distal of said proximal fixation member.

2. A surgical instrument according to claim 1, wherein:
in the loaded configuration, said distal and proximal fixation members are disposed in said distal chamber.

3. A surgical instrument according to claim 1, wherein:
in the loaded configuration, a first section of the surgical mesh is attached to the distal fixation member.

4. A surgical instrument according to claim 1, wherein:
said drive member is movable relative to said elongate member to configure said surgical instrument in a first deployment configuration in which at least a portion of said distal fixation member is positioned outside said distal chamber.

5. A surgical instrument according to claim 4, wherein:
in said first deployment configuration, said drive member is operable to drive said distal fixation member to attach said distal fixation member and said surgical mesh to first tissue at the surgical site by forces applied to said drive member.

6. A surgical instrument according to claim 1, wherein:
said drive member is movable relative to said elongate member to configure said surgical instrument in a second deployment configuration in which said distal fixation member is detached from said instrument and said drive member is in an extended position relative to said elongate member to fully deploy said surgical mesh outside of said distal chamber adjacent the surgical site.

7. A surgical instrument according to claim 6, wherein:
in said second deployment configuration, at least a portion of said proximal fixation member is disposed outside said distal chamber.

8. A surgical instrument according to claim 6, wherein:
the instrument is configurable to the second deployment configuration with the distal fixation member securing the surgical mesh to the first tissue at the surgical site.

9. A surgical instrument according to claim 8, wherein:
said drive member is operable to unfurl the surgical mesh in a controlled manner to deploy the surgical mesh at a desired position adjacent the surgical site.

10. A surgical instrument according to claim 6, wherein:
in said second deployment configuration, said drive member is operable to drive said proximal fixation member through said surgical mesh and into second tissue at the surgical site in order to attach said proximal fixation member and said surgical mesh to the second tissue by forces applied to said drive member.

11. A surgical instrument according to claim 10, wherein:
said second tissue and the section of mesh attached thereto is spaced apart from said first tissue and the section of mesh attached thereto.

12. A surgical instrument according to claim 13 wherein:
said diameter of said elongate member does not exceed 5 mm.

13. A surgical instrument according to claim 1, wherein:
said drive member is rotatable relative to said elongate member, and a longitudinal force applied to said proximal end of said drive member causes rotation and longitudinal translation of said drive member relative to said elongate member.

14. A surgical instrument according to claim 1, wherein:
said drive member and said elongate member are rotatably coupled to each other.

15. A surgical instrument according to claim 22, wherein:
said drive member includes a guide, said elongate member includes a helical groove, and said guide is coupled to said helical groove, the coupling between said guide and said groove causing said drive member to rotate and to translate in a distal direction relative to said elongate member when an axial force is applied to said drive member.

16. A surgical instrument according to claim 1, wherein:
said proximal fixation member includes helical threads.

17. A surgical instrument according to claim 16, wherein:
said distal fixation member includes is threadless.

18. A surgical instrument according to claim 17, wherein:
said distal fixation member includes a barb.

19. A surgical instrument according to claim 1, wherein:
said surgical mesh has a memory for a flat configuration.

20. A surgical instrument according to claim 1, wherein:
said distal chamber includes a closed tubular body and an open distal end, and in said first deployment configuration, said drive member is rotated to deploy said surgical mesh from said open distal end of said distal chamber.

21. A surgical instrument according to claim 1, further comprising:
a spring retraction element biasing said distal end of said drive member into a retracted position within said distal chamber of said interior channel of said elongate member.

22. A method for deploying and securing a surgical mesh at a site of a hernia, the method comprising:
advancing a surgical instrument in a loaded configuration to a location adjacent the hernia, the surgical instrument including an elongate member defining an interior channel extending therethrough to an interior distal chamber with an open distal end, a drive member extending through the channel of the elongate member, the drive member having proximal and distal ends, a proximal fixation member detachably coupled to the distal end of the drive member, and a distal fixation member detachably coupled to the proximal fixation member, wherein the mesh and proximal and distal fixation members are disposed inside the distal chamber of the elongate member in the loaded configuration, and a first section of the surgical mesh is attached to the distal fixation member in the loaded configuration;
first deploying at least a portion of the distal fixation member through the open distal end to a location outside of the elongate member;
first attaching the distal fixation member and the first section of the surgical mesh to first tissue at the site of hernia;
second deploying the surgical mesh through the open distal end to a position adjacent the surgical site and positioning the proximal fixation member to a location outside of the elongate member; and
second attaching the proximal fixation member to a second section of surgical mesh and to second tissue at the site of the hernia.

23. A method according to claim 22, wherein:
the second tissue is offset from the first tissue on opposite sides of the hernia.

24. A method according to claim 23, wherein:
the first tissue is softer than the second tissue.

25. A method according to claim 22, wherein:
in the loaded configuration of the surgical instrument, the mesh is helically coiled within the interior chamber, and the proximal and distal fixation members are aligned in an end-to-end configuration within a cylindrical space defined by the helically coiled mesh distal of the drive member;

26. A method according to claim 25, further comprising:
detaching the distal fixation member from the instrument prior to completely deploying the surgical mesh from the distal chamber.

27. A method according to claim 22, wherein:
said advancing, said first deploying, said first attaching, said second deploying and said second attaching all occur through a 5 mm port.
